(19) United States
(12) Patent Application Publication
(10) Pub. No.: US 2013/0250246 A1
(43) Pub. Date: Sep. 26, 2013

(54) DIRECTIONAL ILLUSIONS AND USES THEREOF

(71) Applicant: Arthur Shapiro, Rockville, MD (US)

(72) Inventor: Arthur Shapiro, Rockville, MD (US)

(73) Assignee: American University, Washington, DC (US)

(21) Appl. No.: 13/775,875

(22) Filed: Feb. 25, 2013

Related U.S. Application Data
(60) Provisional application No. 61/602,137, filed on Feb. 23, 2012.

(51) Int. Cl. A61B 3/032

(52) U.S. Cl. CPC A61B 3/032
USPC 351/246

(57) ABSTRACT

The present invention relates to means for creating a visual illusion of directional movement. In various embodiments, methods for using the directional illusion are provided, such as, for the measurement and/or assessment of visual performance and/or acuity, for evaluating visual performance in various settings, enhancing advertising and marketing by drawing attention to a desired symbol, or enhancing images.

This application claims priority to co-pending U.S. patent application Ser. No. 61/602,137 filed Feb. 23, 2012, which is expressly incorporated by reference herein in its entirety.

The present invention relates to means for creating a visual illusion of movement. In one embodiment, the invention provides methods for using a directional illusion for the measurement and/or assessment of visual performance and/or acuity. In another embodiment, the invention further provides an apparatus or arrangement for evaluating visual performance in various settings. In other embodiments, the directional illusion has other applications, such as enhancing advertising and marketing by drawing attention to a desired symbol; enhancing images, such as personal digital photos and video games.

### BACKGROUND OF THE INVENTION

A visual illusion (also called an optical illusion) is characterized by visually perceived images that differ from objective reality. The information gathered by the eye is processed in the brain to give a perception that does not tally with a physical measurement of the stimulus source. There are three main types: literal optical illusions that create images that are different from the objects that make them; physiological optical illusions that are the effects on the eyes and brain of excessive stimulation of a specific type (brightness, colour, size, position, tilt, movement); and cognitive optical illusions, the result of unconscious inferences.

Motion perception is responsible for a number of sensory illusions. Film animation is based on the illusion that the brain perceives a series of slightly varied images produced in rapid succession as a moving picture. Likewise, when we are moving, as we would be while riding in a vehicle, stable surrounding objects may appear to move. We may also perceive a large object, like an airplane, to move more slowly than smaller objects, like a car, although the larger object is actually moving faster. The Phi phenomenon is yet another example of how the brain perceives motion, which is most often created by blinking lights in close succession. The perception of motion can also be created by what has been termed “reversed phi,” in which two lights of opposite contrast polarity are alternated to create the appearance of motion in the direction opposite to that predicted by Phi. Others have used phi and reverse phi to create the appearance of continual motion (i.e., illusory motion perpetually moves in one direction) by juxtaposing two images and slightly offset negatives (referred to as four-stroke motion) or by inserting a gray frame between two slightly offset lights (referred to as two-stroke motion). In addition, non-continual motion can be created by modulating the luminance of thin lines at edges surrounding objects: if a gray rectangle is bordered on the left by a thin white line and on the right by a thin black line, then modulating the luminance of a field surrounding the rectangle will make the rectangle appear to shift back and forth; i.e. when the surrounding field is bright, the rectangle appears to shift to the right, and when the surrounding field is dark, the rectangle appears to shift to the left. In the latter case, the motion arises even though the rectangle and the edges are physically stationary.

We present displays that lead to the perception of continual motion but do not create changes in physical space—that is, continual motion from physically stationary objects. In other words, the displays combine the perceptual motion found in reverse phi phenomena with the thin edges found in edge motion conditions. The key insight into these conditions is that motion signals can be created by modulating the luminance of thin edges in relation to the phase of luminance modulation of fields that surround the edge. When viewing displays that combine opposite direction motion signals, the visual system will group the display into the perception of a moving object. A motion signal is created by changing the modulation at the edge of the field.

Traditional vision acuity tests have used static optotypes as displays of printed or projected characters, objects, or shapes. Numerous patterns, configurations, and methods for static optotypes have been proposed for testing acuity based upon the ability of a subject to distinguish these various shapes, sizes, contrasts, and colors in tests such as Snellen charts, tumbling “E” arrays (static images of the letter “E” where the static image is also rotated 90 degrees, 180 degrees, and 270 degrees for discernment), Landolt “C” charts, and so on. Certain prior art vision testing patterns use periodic images, such as disks, rectangles, diamonds, etc.; others are quasi-periodic, such as tri-bar, and small checkerboard designs.

While the Landolt “C” chart is the clinical standard for acuity, the familiar Snellen eye testing chart as developed in 1862 using large, black, serifed letters on a white background is the test frequently used for determining visual acuity. The concept of these charts to verify acuity is based upon the patient seeing patterns such as letters or printed images on those charts. Snellen's standard is that a person should be able to see and identify a 3.5 inch letter at a 20 foot distance (that ratio being consistent regardless of its use in the “English” or Metric system). A disadvantage of the Snellen type images is that even defocused letters can still be partially recognized by their blur patterns. Much time is thus wasted as the patient, whose eyes are being tested, attempts to guess the letter. The design of the Snellen chart is further complicated by each letter having a different degree of recognizability and by the tendency of the patient to strain to perceive coherency when trying to identify the letters.

Thus, most visual testing systems in optometry offices use optical devices for measuring acuity; however, one problem often encountered with these devices is how to assess acuity in active environments, such as for sports activities or military training.

### SUMMARY OF THE INVENTION

In one aspect of the invention, a visual directional illusion is provided, in which a displayed object appears to move. The observer's perception of movement of objects on a screen is created by means of changes in temporal luminescence, contrast, width, and temporal phase at the edges of the displayed object and/or the surrounding field.

In another aspect of the invention, a dynamic visual acuity assessment method is provided, where various parameters of the directional illusion are varied, and an observer responds to these changed parameters.

In another aspect of the invention, a method for enhancing a displayed image is provided, where the image exhibits the directional illusion.

### BRIEF DESCRIPTION OF THE FIGURES

FIG. 1 shows a schematic of one embodiment of the invention.

FIG. 2 shows a sine wave representing changes in a characteristic of at least one of the edges, in one embodiment of the invention.

FIG. 3 shows a phase shift between the changes in at least one of the edges and the changes in the surrounding field, in one embodiment of the invention.

### DETAILED DESCRIPTION

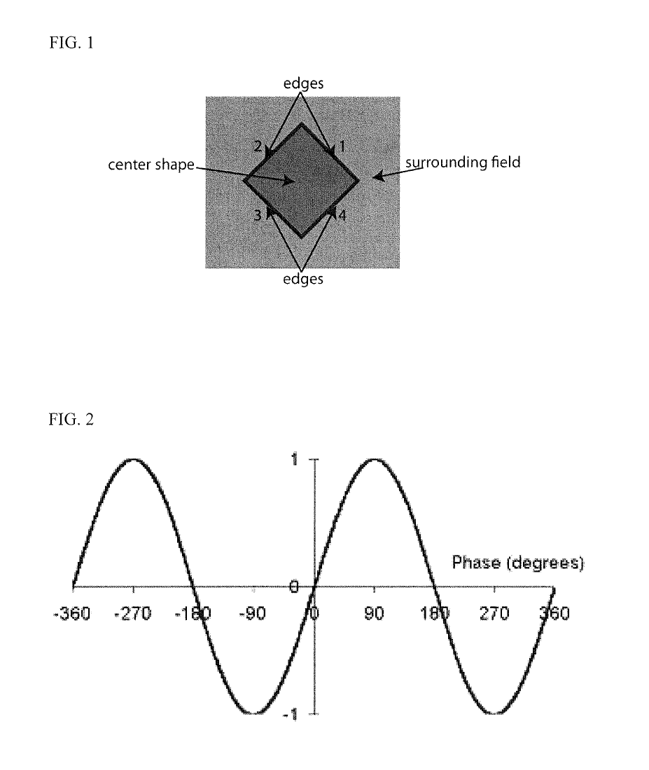

In one aspect of the invention, an optical or visual illusion is provided, which gives the impression of movement to an observer. In one embodiment, the illusion comprises a center shape having edges that border the center shape, and a surrounding field, as shown in FIG. 1. In one embodiment, an illusion of movement is created by small, e.g., 10 degrees at 3 Hz modulation, temporal phase and/or contrast changes in thin edges (e.g., <1 minute of visual angle) surrounding the center shape, which creates an observer's perception of directional movement of the center shape.

The directional illusion will be described where the center shape is a diamond, which is convenient because it has four sides and is oriented obliquely, but other shapes can also be used, for instance an arc-shape can be used to create the appearance of clockwise and counter-clockwise motion. The center shape can be any hue, but the luminance should be between the maximum and minimum luminance of the edges and the surrounding field. Unlike the edges and the surrounding field, the luminance of the center shape does not change over time. The objects of the illusion, i.e., center shape, edges, and surrounding field, can be presented or depicted in any suitable format, such as being displayed on a CRT or LCD monitor.

There are two aspects of the edges which may be changed over time: a) luminance and b) the width of the edges. In one embodiment, the luminance of the edges changes over time such that the edges become light and dark and then repeat. The profile of the luminance change can be described as a sine wave with the luminance on the y-axis and time on the x-axis, as shown schematically in FIG. 2. There are four parameters of the sine wave that can be used to control the light to dark pattern: mean, amplitude, frequency and phase. In one embodiment, the mean luminance level of the edge is substantially similar to the luminance level of the center shape. The amplitude of the sine wave describing the temporal changes in luminance of the edges may be variable. The illusory motion gets stronger as the amplitude gets larger; however, this effect co-varies with the amplitude of the surrounding field, as described below. In various embodiments, the illusion occurs when the frequency of the modulation of the edges is between about 1 Hz and about 8 Hz. Faster rates, e.g., higher frequencies, may also be employed, but in some cases, the frequency is limited by the capabilities of CRT and LCD monitors. Lastly, the phase, which represents the edge changes relative to changes in the surrounding field, may also be manipulated for creating the illusion of directional motion.

As described above, the width of the edges also play a role in the creation of the illusion. The illusion of motion can be perceived when the edges are thin, i.e, <1 min of visual angle. The ability to see the motion when the edges are thin depends upon the observer's visual acuity.

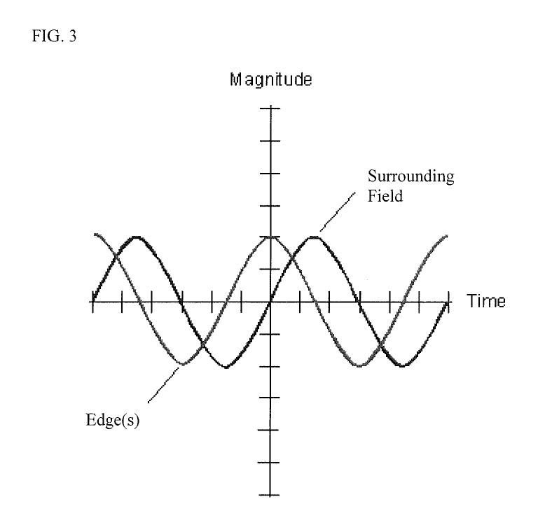

As with the edges, the luminance of the surrounding field changes over time in a sinusoidal fashion, for example, as shown in FIG. 3. There are four parameters for the sine wave that control the light to dark pattern: mean, amplitude, frequency and phase. In one embodiment, as with the edges, the mean luminance level of the surrounding field is substantially similar to the luminance level of the center shape. The amplitude of the sine wave describing the temporal changes in luminance of the surrounding field may be variable. The illusory motion gets stronger as the amplitude gets larger; however, this effect co-varies with the amplitude of the edges. In one embodiment, the frequency of modulation matches the frequency of the edge modulation. Lastly, as described in detail below, the phase, which represents the edge changes relative to changes in the surrounding field, is manipulated to create the illusion of directional motion. In one embodiment, the temporal phase of the surround is defined as 0 degrees, and the bottom edges modulate at −90 degrees while the top edges modulate at +90 degrees. In this case, the apparent motion inside the center object is upward. If the temporal phase of the surround is shifted to 180 degrees, the motion will be downward. The size of the surrounding field does not matter, as long as its spatial extent is larger than the center field and the edges.

The center object, which will be perceived as moving by the described changes in edge and surrounding field, can be any shape, such as geometric figures or real-world objects, such as a picture of a baseball or a football.

The perception of directional motion can be manipulated by varying the parameters described above, for example, by varying the phase and/or contrast between the edges and the surrounding fields. For example, by changing the temporal phase relationship between the edges and the surrounding field, the center shape will appear to be moving up, down, left, right, inward or outward. In the case of the diamond shown in FIG. 1, if the phase of the luminance changes of the edges are varied relative to the changes in the surrounding field, a directional illusion is created in one of six directions, as shown in the table below.

| Perceived | Phase of edge modulation relative to | | | |
|---|---|---|---|---|
| direction of | the surrounding field modulation | | | |
| the diamond | Edge 1 | Edge 2 | Edge 3 | Edge 4 |
| Upward | Lead 90° | Lead 90° | Trail 90° | Trail 90° |
| Downward | Trail 90° | Trail 90° | Lead 90° | Lead 90° |
| Left | Trail 90° | Lead 90° | Trail 90° | Lead 90° |
| Right | Lead 90° | Trail 90° | Lead 90° | Trail 90° |
| Inward | Trail 90° | Trail 90° | Trail 90° | Trail 90° |
| Outward | Lead 90° | Lead 90° | Lead 90° | Lead 90° |

In the above table, a complete cycle of the sine wave represents 360 degrees. Thus, a variance in the phase between the edge and surrounding field of 90 degrees represents a quarter of a cycle. In other words, the cyclic variation of the luminance of the edge is shifted either to the left, and occurs earlier in time, or is shifted to the right, and occurs later in time, in relation to the modulation of the surrounding field.

In one embodiment, multiple center shapes, each with edges, can be placed in the surrounding field. For example, the center shapes may be arranged in a circular pattern, such that creating the illusion of movement of the center shapes creates the illusion that the circular pattern is itself rotating, where the rotation can be created to be either clockwise, counterclockwise, or alternating between the two directions.

In another aspect of the invention, the directional illusion is used as a simple, definitive assay for the measurement of visual performance, such as sensitivity to contrast and visual acuity. In one embodiment, to test for visual acuity, the width of the edges can be changed until the observer cannot correctly identify the direction of motion of the shape. Alternatively, the observer can move away from the screen until he/she cannot correctly identify the direction of motion. Acuity can be measured in general settings, such as a doctor's office, or in dynamic environments such as in a video game or in an active environment (such as with sports or military) where it might be advisable to measure acuity while observers participate in an activity; or mass screening in public health situations. In addition, the display of the directional illusion can be presented to different ocular locations to test for visual acuity in the visual periphery.

The use of a circular array of center shapes, as described above, may be used to compare acuity of gaps in surfaces (like measured in the Landolt c acuity) and can also be used as an alternative diagostic.

In one embodiment, the described directional illusion permits an accurate determination of acuity and allows patients to more accurately perceive visual acuity than they can by use of static reflected or projected letters, symbols, or shapes, as used by the Snellen and similar tests. In an acuity test, a subject may be asked to indicate when the movement illusion is perceived. In one embodiment, the parameters of the directional illusion can be varied, as described above, until the subject indicates the perception of movement in the image. Alternatively, the parameters of the directional illusion can remain fixed, and the distance between the subject and the device displaying the directional illusion can be varied.

The acuity threshold, which is the perception of motion from a specific distance, correlates to the specific visual acuity. In viewing directional illusion images, the subject either sees the motion of the image because the viewing distance is close enough and the acuity is sufficient, or the patient does not see the motion because the distance is too far and the acuity is insufficient. Unlike the Snellen test, the subject does not need to be able to read English letters to identify the acuity threshold, to identify the direction of motion.

In one embodiment, the visual acuity test is viewed on a standard computer monitor or projected image at distances equivalent to and corresponding to the Snellen test.

In addition to the use of the described directional illusion in traditional-type visual assessments, the illusion can also be used in various settings. For example, the directional illusions could be used as a screening device for detecting visual problems over the internet, or for mass screening. One issue in public health is to assess when vision is poor in group situations quickly and efficiently (often, this form of assessment uses letters or illiterate tests). In one embodiment, the individuals to be assessed could move towards the screen displaying the illusion and state when they see the movement. The distance from the screen, size of the image, and other pertinent variables may be recorded.

The directional illusion could also be used in non-clinical settings. Human vision is an information-processing task. The human eyes are capable of looking at what is where, but the brain processes and generates a representation of this information in its profusion of color, form, motion and detail. The central vision (center of our retina) has the highest visual acuity and discriminative vision. Visual acuity decreases with distance from the fovea (the center of the retina) to the periphery. The combined field of view of our both eyes is approximately 180° with a 120° area of overlap. In general, the periphery is a larger low resolution field, and the central is a smaller high resolution field.

The central area or fovea subtends only for 2.5° of our visual field, but our head movements coupled with rapid saccadic eye movements give the impression that the combined field of view has a resolution similar to that of the foveal resolution (high resolution). The fovea also uses these saccadic eye movements to acquire peripheral targets. For example, if a viewer fixates foveal vision at the center of a large web page, the viewer will experience the illusion that the entire page is equally legible. It is only when we maintain our focus at the center of the web page and do not shift our eyes to the edge that we realize that the periphery is illegible.

By suppressing our natural tendency to turn our head or eyes, the peripheral regions of the retina can be trained to identify objects, thus improving the peripheral vision. This can be achieved by instructing the subject to stare at a visual marker that is intended for the subject to focus on using the central vision. While the subject is looking directly at the visual marker, a peripheral target is also displayed on the screen. The peripheral target is intended for identification using the peripheral vision of the subject, while the subject is directly looking at the visual marker. Identification of peripheral targets, in general, refers to recognizing characteristics of the target (i.e., visually discernable characteristics) in addition to detecting the presence of target. In the present case, the peripheral target is the described directional illusion, and the subject's task is to identify movement. This practicing task trains the subject to use visual activities to identify objects using the peripheral vision. This task also serves the purpose of assessing the subject's peripheral vision. In a further embodiment, the direction of movement of the illusion may be changed during testing, and the subject is asked to identify these changes.

The subject can engage in these visual activities using a portable device. For example, the visual mark and peripheral targets can be displayed in a video or an image on a computer screen, a laptop computer screen, a television set or screen, and/or portable device including but not limited to a mobile phone, an MP3 player, a Blackberry, a Palm Treo, a handheld computer, a head-mounted unit, and/or an iPhone, etc.

As further examples, the images of the directional illusion can be placed in sports video goggles or other head-mounted displays and used in various visual performance assessments and/or visual training. In another embodiment, monitors displaying the illusion can be placed in different locations around a testing environment, and observers can label the direction of motion in the displays. For example, a method for improving a subject's peripheral vision is to present the directional illusion on a display screen, where the displayed directional illusion is in the subject's peripheral vision. The subject would then be asked to correctly identify motion of the illusion using peripheral vision.

In other embodiments, the directional illusion could be inserted into a video game in which rewards, targets, or movement around or at an object depends upon correctly identifying the direction of motion in a diamond.

In further embodiments, the directional illusion can be used to customize displays of personal images. For example, the images could be provided by a customer, and the images could be modified or enhanced to introduce the illusion of movement into the image.

In yet further embodiments, the directional illusion can be used as a technique to direct attention to an image or as a marketing strategy.

1. A method for creating an illusion of movement, the method comprising
(a) displaying an image comprising a center shape, the center shape having a plurality of edges, and a surrounding field, wherein the edges have a first luminance and width, and the surrounding field has a second luminance,
(b) varying at least one of the first luminance or width of at least one of the plurality of edges of the center shape, such that the variation is sinusoidal and having a mean, an amplitude, a frequency and a first phase, and
(c) co-varying the second luminance of the surrounding field, such that variation is sinusoidal and having a mean, an amplitude, a frequency and a second phase, wherein the illusion of movement is created when the first phase is different from the second phase.

2. A method for testing visual acuity, the method comprising
displaying the illusion of claim 1 to a subject,
receiving feedback from the subject as a result of viewing the illusion, and
using the received feedback to ascertain visual acuity of the subject.

3. A method for testing visual acuity, the method comprising:
(a) displaying to a subject, an image comprising a center shape, the center shape having a plurality of edges, and a surrounding field, wherein the edges have a first luminance and width, and the surrounding field has a second luminance,
varying at least one of the first luminance or width of at least one of the plurality of edges of the center shape, such that the variation is sinusoidal and having a mean, an amplitude, a frequency and a first phase, and
co-varying the second luminance of the surrounding field, such that variation is sinusoidal and having a mean, an amplitude, a frequency and a second phase,
(b) creating a difference between the first phase and the second phase, and
(c) ascertaining whether the subject can perceive movement in the center shape.

4. The method of claim 3 wherein in step (b), the difference between the first phase and the second phase is varied systematically until the subject perceives motion.

5. The method of claim 3 wherein the distance between the subject and the image is systematically varied until the subject perceives motion.

6. A method for enhancing visual performance, the method comprises
(a) displaying to a periphery of a subject, an image comprising a center shape, the center shape having a plurality of edges, and a surrounding field, wherein the edges have a first luminance and width, and the surrounding field has a second luminance,
varying at least one of the first luminance or width of at least one of the plurality of edges of the center shape, such that the variation is sinusoidal and having a mean, an amplitude, a frequency and a first phase, and
co-varying the second luminance of the surrounding field, such that variation is sinusoidal and having a mean, an amplitude, a frequency and a second phase,
(b) creating a difference between the first phase and the second phase, and
(c) ascertaining whether the subject can perceive movement in the center shape.
